(19) United States
(12) Patent Application Publication
(10) Pub. No.: US 2026/0185029 A1
(43) Pub. Date: Jul. 2, 2026

(54) CELL CULTURE MEMBRANE AND PRODUCTION METHOD

(71) Applicants: HACETTEPE UNIVERSITESI, Ankara (TR); CANKAYA UNIVERSITESI, Etimesgut/Ankara (TR)

(72) Inventors: Pervin Rukiye DINCER, Ankara (TR); Ismail UYANIK, Ankara (TR); Samet AKAR, Ankara (TR); Harun ARTUNER, Ankara (TR); Nilufer DUZ, Ankara (TR); Yasin GULSUM, Ankara (TR); Waleed Jehad Mohammad ODEIBAT, Riyadh (SA)

(73) Assignees: HACETTEPE UNIVERSITESI, Ankara (TR); CANKAYA UNIVERSITESI, Ankara (TR)

(21) Appl. No.: 19/124,905

(22) PCT Filed: Oct. 25, 2023
(86) PCT No.: PCT/TR2023/051193

§ 371 Date: Apr. 28, 2025

(30) Foreign Application Priority Data
Oct. 27, 2022 (TR) 2022/016333

(51) Int. Cl. C12M 1/12

(52) U.S. Cl. CPC C12M 25/04

(57) ABSTRACT

Provided is a cell culture membrane for use during a cell stretching process that stresses the cell to induce mechanical signaling and thus biochemical signals in the cell. A production method for the cell culture membrane includes: providing a mold assembly with a lower plate containing pin slots, an upper plate placed on the lower plate and including at least one window, pins to be placed in said pin slots, and a mold to be placed between the pins remaining in said window; placing pins in the pin slots remaining in the said window and placing a mold of primary length in the window; pouring the polydimethylsiloxane-solidifier mixture into the window in an amount that exceeds the primary length; eliminating the air bubbles in the mixture; baking the mold assembly in which said mixture is located, and then leaving the assembly to cool and removing it from the mold assembly.

### TECHNICAL FIELD

The invention relates to a cell culture membrane for use during a cell stretching process that creates stress by applying stretching to the cell to trigger mechanical signaling in the cell and thus biochemical signals.

### STATE OF THE ART

Cell stretching devices have long been used to detect the behavior of cells under different stresses. In these devices, tensile force is applied by various drive elements to a membrane where cell culture is maintained and accordingly, the membrane stretches. With the stretching that occurs in the membrane, the behavior of the cells under mechanical loading is simulated. Flexible structures based on polydimethylsiloxane (PDMS) are generally preferred as membranes.

WO2011143294A2 relates to an upper plate defining one or more cell culture plate wells, one or more flexible cell culture membranes operatively coupled with the upper plate to form liquid-tight base in cell culture plate wells; and a pin plate under the upper plate having one or more flexible cell culture membranes there between; and the pin plate relates to a dynamic in vitro scanning device having one or more pin elements associated with one or more cell culture plate wells.

IN251541B relates to a process that can make molded PDMS membranes. After the PDMS is mixed with a Dow mixture, it is compressed between the two foils and reaches the required thickness by applying two tons of pressure on the foil, then the metal blocks are mounted with pressure and the mold is exposed to a certain temperature. This process is repeated 5 times for membrane production.

U.S. Pat. No. 6,048,723A relates to a cell culture plate having a flexible membrane sandwiched between a base and a body. The document states that if further pre-stretching of the membrane is desired, a tongue/groove arrangement may be included in the single-bowl culture plate design.

The common problem of the studies mentioned here is that the force generated during the stretching movement is not uniformly distributed. Therefore, obtaining standard results is prevented at all points of the surface during the cell stretching process. In addition, especially the high stretching force on the stretching points often causes the membrane to rupture. On the other hand, uneven stress distribution reduces the reliability and repeatability of the experiments.

All the problems mentioned above have made it necessary to make an innovation in the relevant field as a result.

### OBJECTS AND BRIEF DESCRIPTION OF THE INVENTION

The main object of the invention is to reveal the structure of a cell culture membrane that makes uniform stretching force distribution possible during the stretching process.

The object of the invention is to reveal the structure of a method that can be easily adapted for the production of different-sized cell culture membranes.

The present invention relates to the production method for a cell culture membrane to provide the aforementioned requirements. Accordingly, the present invention comprises the steps of providing a mold assembly with a lower plate containing multiple pin slots on its surface, an upper plate placed on the lower plate and comprising at least one window, pins to be placed in said pin slots and a mold to be placed between the pins remaining in said window, placing pins in the pin slots remaining in the said window and placing a mold of primary length in the window so that it is between the pins, pouring the polydimethylsiloxane-solidifier mixture mixed between 10/1.0 and 10/0.5 by weight into the window in an amount that exceeds the primary length in the window, eliminating the air bubbles in the mixture, baking the mold assembly in which said mixture is located, and then leaving the assembly to cool and removing it from the mold assembly.

In one embodiment of the invention, especially in the mold assembly arranged for polygonal membranes, the pin slots are provided only in the edge plane, pin openings are not included in the corners, and accordingly, the corner stretching is completely eliminated.

### DEFINITIONS OF FIGURES DESCRIBING THE INVENTION

The figures and related descriptions used to better describe the device developed by this invention are as follows.

FIG. 1. Isometric image of the mold assembly prepared for the method subject to the invention

FIG. 1a. The isometric image of the mold in FIG. 1

FIG. 1b. Isometric view of the upper plate in FIG. 1

FIG. 2. Isometric image of the membrane prepared for the method subject to the invention

FIG. 3. Simulation results showing the stress distribution of the membrane prepared by the method of the invention

FIG. 3a. Simulation results showing the stress distribution of a membrane prepared by standard methods

### DEFINITIONS OF COMPONENTS/PIECES/PARTS OF THE INVENTION

In order to better explain the device developed by this invention, the parts and pieces in the figures are numbered and the corresponding numbers are given below.
1. Mold assembly
10. Lower plate
11. Pin slot
12. Lower connection opening
13. Connection element
20. Upper plate
21. Window
22. Upper connection opening
30. Mold
40. Connection element
P. Pin
L. Primary length
100. Membrane
101. Base
102. Wall
103. Pin opening

### DETAILED DESCRIPTION OF THE INVENTION

The subject matter of the invention relates to a cell culture membrane (100) for use during a cell stretching process that stresses the cell by applying stretching to the cell to trigger mechanical signaling in the cell and thus biochemical signals.

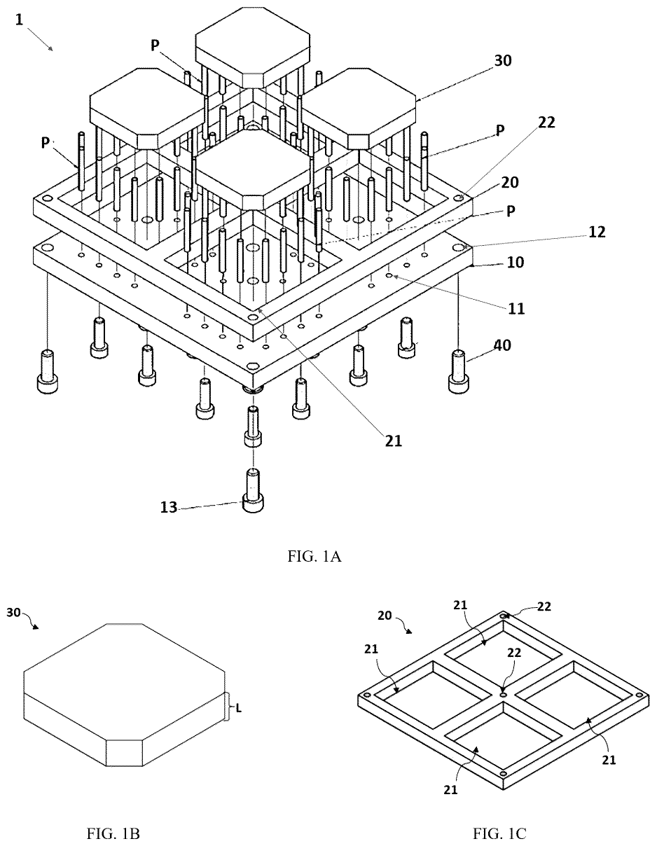

With reference to FIG. 1, a specific mold assembly (1) is provided for the method of the invention.

The mold assembly (1) is configured on a lower plate (10). The lower plate (10) is provided in a planar manner. Multiple pin slots (11) are arranged on the surface of the lower plate (10). The pin slots (11) are preferably circular inlets. Here, the pin slots (11) are provided in the form of a groove and do not reach the other surface of the lower plate (10) and thus do not cause the mixture to exit the mold assembly (1) during the mixture application to be described later.

An upper plate (20) is placed on the lower plate (10). The upper plate (20) is provided in a planar manner. The upper plate (20) mentioned herein comprises at least one window (21). The window (21) is an opening above the upper plate (21). Here, the shape of the opening, that is, the window (21), determines the geometry of the peripheral edges of the membrane (100) to be obtained. Preferably, the windows (21) are provided in a rectangular shape. As can be seen in FIGS. 1 and 1b, the upper plate (20) may also comprise multiple windows (21).

The said pin slots (11) are formed in the position to remain inside the windows (21) when the upper plate (20) is placed on the lower plate (10). That is, the said pin slots (21) are accessible from the windows (21).

The said pin slots (11) are provided along the edges of the said window, with more than one on each edge. Accordingly, the pin openings (103) of the membrane (100) to be obtained will be provided sequentially at the edges, which will ensure a more homogeneous distribution of the force when the tensile force is applied to the membrane (100) from the pin openings (103).

Preferably, the pin slots (11) are adjusted so that they do not coincide with the corner points in the polygonal windows (21), since the tensile force applied from the corner points negatively affects the homogeneous distribution of the tensile force.

The lower plate (10) and the upper plate (20) are preferably fixed to each other by bolt or screw-like connection elements (40) after the window (21)-pin slot (11) described above is placed on top of each other in a position to be positioned.

The mold (30) shown in FIG. 1a is positioned between the pins (P) placed in the pin slots (11) during production. For this reason, it cannot have a large base or ceiling area from the area created by the said pin slots. The mold (30) is responsible for ensuring that the base (101) of the membrane (100) is formed.

The mold (30) can be produced in different sizes according to the desired dimensions of the membrane (100) and the geometry of the membrane (100) will change according to the dimensioning of the mold (30).

The lower plate (10), the upper plate (20) and the mold (30) are produced by the milling method using CNC machine tools. The outer frame of the mentioned elements is produced by the wire erosion cutting method in order to minimize distortions and sprains. In addition, polishing was carried out in order to improve the surface quality of the mentioned elements, and then anodized coating was applied.

For the production of the membrane (100) subject to the invention, it is first necessary to produce the said mold assembly (1). First of all, the elements of the mold assembly (1) are cleaned with preferably alcohol-based cleaning products and it is ensured that the wastes and dust particles from the old production are removed.

As mentioned above, first the lower plate (10) and the upper plate (20) are connected to each other by means of screws, and then the pins (P) are placed vertically in the pin slots (11).

The mold (30) suitable for the membrane (100) to be produced is positioned between the said multiple pins (P).

Then, the fluid polydimethylsiloxane-solidifier mixture is filled into the said window (21). It should be mixed with the solidifier and then degassed initially. The minimum amount of mixture to be added here is the amount that will ensure that the length of the mixture filled into the window (21) is longer than the primary length (L) of the mold (30). If less than this amount of mixture is added, the membrane (100) base (103) will not be formed. As can be understood from this amount, the window volume differs according to mold size or volume.

During the pouring and/or preparation of the mixture, bubbles are formed in the mixture. The presence of bubbles creates a disadvantage for an appropriate stretching force distribution. For this reason, it is necessary to eliminate bubbles.

For bubble elimination, the mold assembly (1) in which said mixture is poured is exposed to high air pressure. The pressure is applied between −0.05 and −0.07 MPa. The mixture, which is free from bubbles, is subjected to baking.

Here, baking is preferably provided between 4 hours and 55-60° C. It is advantageous to keep the temperature constant during baking.

It is important to wait for the mold assembly and membranes (100) to cool after the baking process is completed.

The pins (P) are removed from the cooling mold assembly (1) first. Preferably, with the help of a spatula, the lower plate (10) and the upper plate (20) are separated from each other and the membrane (100) is ready for use.

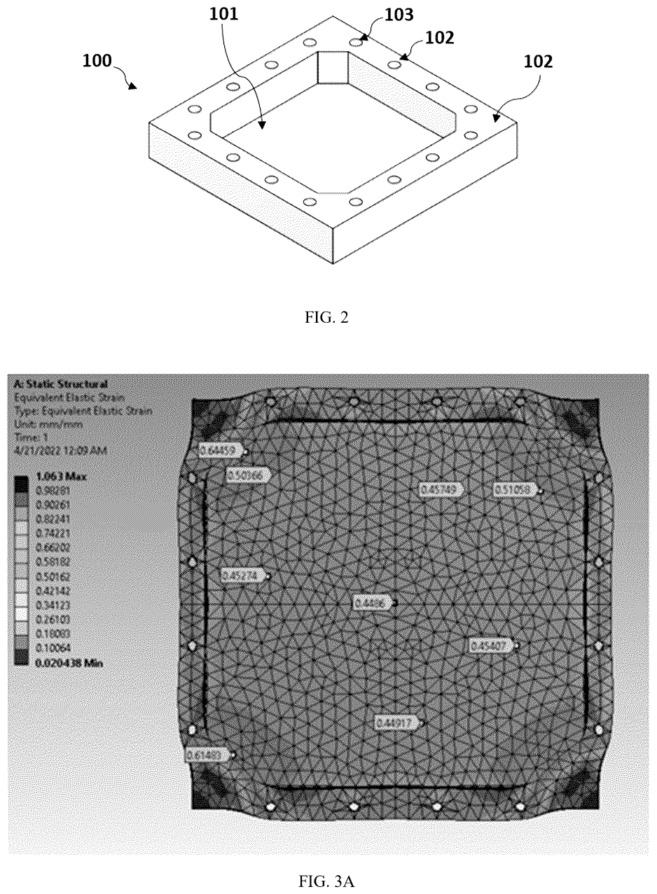

Referring to FIG. 2, said membrane (100) comprises a base (103) and a wall (102) extending perpendicularly from said base (103), and on this wall (102) there are pin openings (103) forming a channel between the two surfaces. As previously mentioned, the geometry of the outer edges of the membrane (100) is formed according to the geometry of the window (21).

For the membrane (100) obtained here to provide a homogeneous tensile force during the stretching process, the ratio of the polydimethylsiloxane-solidifier mixture from which the membrane (100) is obtained is of great importance. Here, the solidifier is a special material used for polydimethylsiloxane in the structure of the membrane (100) where the cells are cultured. The solidifier hardens the polydimethylsiloxane by cross-linking with the polymeric matrix of the polydimethylsiloxane.

Within the scope of this study, it has been observed that the polydimethylsiloxane-solidifier mixtures that will provide the desired homogeneous force distribution should vary between 10/1.0 and 10/0.5 by weight. The most optimal result was obtained in 10/0.75 polydimethylsiloxane-solidifier mixture.

In addition, it has been observed that the temperature applied during baking and the duration of application of this temperature are of great importance in the performance of the membrane (100).

The tensile tests for the membranes (100) obtained as a result of the production of polydimethylsiloxane-solidifier mixtures at various mixture ratios at various temperatures are given in Table 1. If the membrane (100) is working, the “V” sign is used, and if there is a tear in the membrane, the “X” sign is used.
Heat treatment test results at 60° C. for 4 hours

Stretching Rates

| PDMS/Solidifier | 10% | 15% | 20% |
|---|---|---|---|
| 10/1 | X | X | X |
| 10/0.75 | ✓ | ✓ | ✓ |
| 10/0.5 | ✓ | X | X |

30 minutes 80° C.+60 minutes 100° C. heat treatment test results

TABLE 1
PDMS/Solidifier Ratios and Heat Treatment Times Tested

Stretching Rates

| PDMS/Solidifier | 10% | 15% | 20% |
|---|---|---|---|
| 10/1 | X | X | X |
| 10/0.75 | ✓ | ✓ | X |
| 10/0.5 | ✓ | X | X |

As can be seen, tearing becomes inevitable, especially when the polydimethylsiloxane-solidifier ratio exceeds 10/0.75. In addition, the best results at both temperatures were obtained with a mixture of 10/0.75 by weight, but the membrane obtained with this mixture showed 20% stretch ruptures when baked at 80° C. for 30 minutes+100° C. heat treatment for 60 minutes. The optimum solution was obtained by heat-treating the mixture at a ratio of 10/0.75 for 4 hours at 60° C.

The Uniformity Index value of this optimum solution was examined. The uniformity index refers to how linear and evenly distributed the stress applied to the membrane 100 is to the surface. It is the ratio of the total area of the examined membrane (100) to the uniform stretching area. The uniformity index cannot exceed 1.0. The uniformity index calculation of the membrane where cell culture is performed is determined as follows.

$$Total area of membrane = 2650 {mm}^{2} ( under 20 \% stress )$$

$$Uniformity index of 80 \% and above = 1405 {mm}^{2} ( under 20 \% stress )$$

$$Uniformity index = 1405 / 2650 = 0.53$$

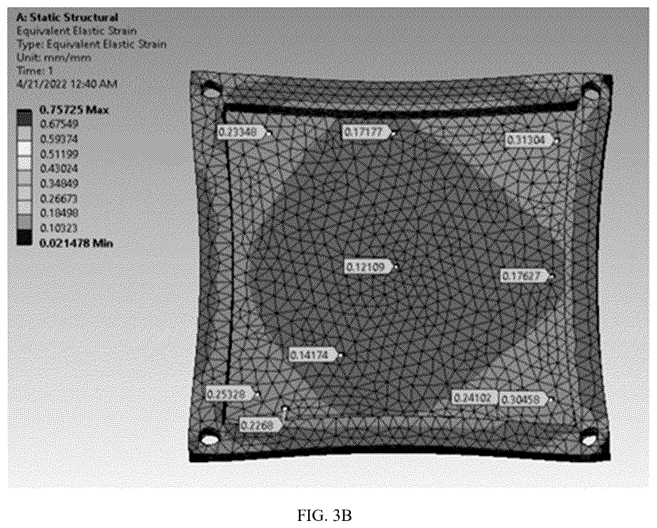

Referring to FIGS. 3 and 3a, a visual representation of the finite element analysis of a membrane (100) and a standard membrane (100) cured at 60° C. for 4 hours of the mixture at the rate of 10/0.75 by weight, respectively, was made in the aforementioned images. As can be seen here, while very close values were reached on the surface of the membrane (100) subject to the invention, high values were observed in the other membrane, especially in the corners, compared to the remaining part.

What is claimed is:

1. A production method for a cell culture membrane, comprising:
providing a mold assembly, wherein the mold assembly comprises a lower plate comprising a plurality of pin slots on a surface of the lower plate, an upper plate disposed on the lower plate and comprising at least one window, pins for placement in the plurality of pin slots, and a mold for placement between the pins remaining in the at least one window,
inserting the pins in the plurality of pin slots remaining in the at least one window and inserting the mold in the at least one window with a primary length between the pins,
pouring a polydimethylsiloxane-solidifier mixture mixed between 10/1 and 10/0.5 by weight into the at least one window in an amount exceeding the primary length in the at least one window,
eliminating air bubbles in the polydimethylsiloxane-solidifier mixture, baking the mold assembly comprising the polydimethylsiloxane-solidifier mixture, allowing the mold assembly to cool, and removing the cell culture membrane from the mold assembly.

2. The production method according to claim 1, wherein the polydimethylsiloxane-solidifier mixture comprises 10/0.75 by weight of polydimethylsiloxane-solidifier.

3. The production method according to claim 1, wherein the air bubbles are eliminated by exposing the polydimethylsiloxane-solidifier mixture to pressure.

4. The production method according to claim 3, wherein the polydimethylsiloxane-solidifier mixture is exposed to a pressure of −0.05 MPa to −0.07 MPa.

5. The production method according to claim 1, wherein the mold assembly comprising the polydimethylsiloxane-solidifier mixture is baked between 55° C. and 60° C.

6. The production method according to claim 5, wherein the mold assembly comprising the polydimethylsiloxane-solidifier mixture is baked at 60° C.

7. The production method according to claim 1, wherein the polydimethylsiloxane-solidifier mixture is baked at a constant temperature.

8. The production method according to claim 1, wherein the lower plate is provided with more than one pin slot on each edge of the at least one window.

9. The production method according to claim 1, wherein the at least one window is in a form of a polygon.

10. The production method according to claim 1, wherein the at least one window is in rectangular form.

11. The production method according to claim 8, wherein the plurality of pin slots are provided only on edges of the at least one window.

12. A cell culture membrane produced by the production method according to claim 1.

13. The production method according to claim 2, wherein the mold assembly comprising the polydimethylsiloxane-solidifier mixture is baked between 55° C. and 60° C.

14. The production method according to claim 3, wherein the mold assembly comprising the polydimethylsiloxane-solidifier mixture is baked between 55° C. and 60° C.

15. The production method according to claim 4, wherein the mold assembly comprising the polydimethylsiloxane-solidifier mixture is baked between 55° C. and 60° C.

16. The production method according to claim 5, wherein the polydimethylsiloxane-solidifier mixture is baked at a constant temperature.

17. The production method according to claim 6, wherein the polydimethylsiloxane-solidifier mixture is baked at a constant temperature.

18. The production method according to claim 9, wherein the plurality of pin slots are provided only on edges of the at least one window.

19. The production method according to claim 10, wherein the plurality of pin slots are provided only on edges of the at least one window.

20. The cell culture membrane according to claim 12, wherein in the production method, the polydimethylsiloxane-solidifier mixture comprises 10/0.75 by weight of polydimethylsiloxane-solidifier.
